(19) United States
(12) Patent Application Publication
(10) Pub. No.: US 2015/0132440 A1
(43) Pub. Date: May 14, 2015

(54) DIPEPTIDE AND TRIPEPTIDE COMPOSITIONS AND USES

(71) Applicant: 4141 HOLDINGS, LLC, Weston, FL (US)

(72) Inventor: John H Owoc, Weston, FL (US)

(21) Appl. No.: 14/459,173

(22) Filed: Aug. 13, 2014

Related U.S. Application Data
(60) Provisional application No. 61/865,405, filed on Aug. 13, 2013.

(51) Int. Cl. A23L 1/305; C07K 5/068; A23L 2/38; A23L 1/303; A23L 2/66; A23L 2/395; C07K 5/083; A23L 1/302

(52) U.S. Cl. CPC A23L 1/3053; C07K 5/0808; C07K 5/06086; A23L 1/302; A23L 1/303; A23L 2/66; A23L 2/395; A23L 2/38; A23V 2002/00
USPC 426/73; 530/331; 562/560; 426/656; 426/548; 426/72

(57) ABSTRACT

A composition of dipeptides or tripeptides or ester derivatives may be used as a nutritional supplement and to modulate expression of genes associated with healthy maintenance and regulation of muscular and neurological tissue. The compositions include dipeptides and/or tripeptides of branched chain amino acids and optionally include adjuvants, anti-aging compounds and may be provided in a dissolvable powder or liquid form.

### CROSS-REFERENCE TO RELATED APPLICATIONS

This application claims priority to U.S. Provisional Application Ser. No. 61/865,405 filed on Aug. 13, 2013 the contents of which are hereby incorporated in their entirety.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

Not Applicable

### BACKGROUND OF THE INVENTION

1. Field of the Invention

The present invention relates to compositions of dipeptides and tripeptides and their uses. More particularly, the invention relates to dipeptides and tripeptides including valine, leucine, and isoleucine. The compositions may be optimized for ingestion as a ready-to-drink liquid, a powder, sublingual gels, and injectable liquids.

2. Description of the Related Art

A branched-chain amino acid (BCAA) is an amino acid having aliphatic side-chains with a branch (a carbon atom bound to more than two other carbon atoms). Among the proteinogenic amino acids, there are three BCAAs: leucine, isoleucine, and valine. The BCAAs are among the nine essential amino acids for humans, accounting for 35% of the essential amino acids in muscle proteins and 40% of the preformed amino acids required by mammals (“Exercise Promotes BCAA Catabolism: Effects of BCAA Supplementation on Skeletal Muscle during Exercise,”) J. Nutr. 134 (6)).

A dipeptide is two amino acids linked via a peptide bond (also called amide bond), which is a covalent bond formed between two amino acids when the carboxylic group from one amino acid reacts with the amino group from the other amino acid. Likewise, a tripeptide is a peptide consisting of three amino acids joined by peptide bonds.

Six amino acids are taken into muscle tissue for energy including alanine, aspartate, glutamate, and the three BCAAs. The BCAAs have the greatest metabolic potential for energy use in muscle. Muscle tissue has 60% of the specific enzymes needed for oxidation (burning) of amino acids for energy, specifically BCAA's. In essence muscle is designed to burn BCAA amino acids for energy. During exercise, the body uses BCAA's as energy. The longer and harder the workout, the more BCAAs are used in muscle for energy. It is estimated that 3% to 18% of all workout energy is provided by the BCAA's, while some consider these percentages to be conservative.

Shimizu, et al. examined the effects of BCAAs on the development of azoxymethane (AOM)-initiated colonic premalignant lesions in C57BL/KsJ-db/db (db/db) mice that were obese and had hyperinsulinemia. BCAA supplementation in diet improves insulin resistance and inhibits the activation of the IGF/IGF-IR axis, thereby preventing the development of colonic premalignancies in an obesity-related colon cancer model that was also associated with hyperlipidemia and hyperinsulinemia. BCAA's, therefore, may be a useful chemoprevention modality for colon cancer in obese people. (Clin Cancer Res, 2009 May 1; 15(9):3068-75).

A review by Ohtani, et al. discusses some of the beneficial effects of a dietary amino acid supplement on muscle function, fatigue, and recovery in exercising athletes. One beneficial effect of the amino acid supplement was a quicker recovery from the muscle fatigue that followed eccentric exercise training. A dose-response study of the amino acid mixture at 2.2, 4.4, and 6.6 g/d for 1 month showed that, at the highest dose, indices of blood oxygen-carrying capacity were increased and those of muscle damage were decreased. When the amino acid mixture was given for 90 days to elite rugby players during training at a dose of 7.2 g/d, a blood-component analysis indicated improvements in the oxygen-carrying capacity of the blood. Together, the studies suggest that the amino acid supplement contributed to an improvement in training efficiency through positive effects on muscle integrity and hematopoiesis. (“Amino Acid Mixture Improves Training Efficiency in Athletes,”. Nutr. February 2006 vol. 136 no. 2 538S-543S).

It is known that BCAA oxidation is promoted by exercise. The mechanism responsible for this phenomenon is attributed to activation of the branched-chain α-keto acid dehydrogenase (BCKDH) complex, which catalyzes the second-step reaction of the BCAA catabolic pathway and is the rate-limiting enzyme in the pathway. This enzyme complex is regulated by a phosphorylation-dephosphorylation cycle. The BCKDH kinase is responsible for inactivation of the complex by phosphorylation, and the activity of the kinase is inversely correlated with the activity state of the BCKDH complex, which suggests that the kinase is the primary regulator of the complex.

Shimomura, et al. found that administration of ligands for peroxisome proliferator-activated receptor-α (PPARα) in rats caused activation of the hepatic BCKDH complex in association with a decrease in the kinase activity, which suggests that promotion of fatty acid oxidation upregulates BCAA catabolism. Long-chain fatty acids are ligands for PPARα, and the fatty acid oxidation is promoted by several physiological conditions including exercise. These findings suggest that fatty acids may be one of the regulators of BCAA catabolism and that the BCAA requirement is increased by exercise. Furthermore, BCAA supplementation before and after exercise has beneficial effects for decreasing exercise-induced muscle damage and promoting muscle-protein synthesis; this suggests the possibility that BCAAs are a useful supplement in relation to exercise and sports. (“Exercise Promotes BCAA Catabolism: Effects of BCAA Supplementation on Skeletal Muscle during Exercise,” J. Nutr. Jun. 1, 2004 vol. 134 no. 6 1583S-1587S).

A study by Greer investigated whether BCAA supplementation attenuates indirect indicators of muscle damage during endurance exercise as compared with an isocaloric, carbohydrate (CHO) beverage or a noncaloric placebo (PLAC) beverage. The data suggested that BCAA supplementation attenuates muscle damage during prolonged endurance exercise in untrained college-age men. CHO ingestion attenuates CK activities at 24 and 48 hour postexercise as compared with a placebo beverage. (Greer, B., J. Woodard, J. White, E. Arguello, and E. Haymes. “Branched-chain amino acid supplementation and indicators of muscle damage after endurance exercise,” International Journal of Sport Nutrition and Exercise Metabolism (2007) 17:6, 595-607).

The protective effects of amino acids against H₂O₂-induced oxidative stress were investigated by Shigeru Katayama and Yoshinori Mine (J. Agric. Food Chem., 2007, 55 (21), pp 8458-8464) in an in vitro assay using human intestinal epithelial cells. Caco-2 cells were pretreated with amino acids (1, 2, and 5 mM) for 2 hours and then stimulated with 1 mM H₂O₂ for 6 hours. The secretion of IL-8, a proinflammatory mediator, was determined by ELISA as an indicator of tissue oxidative stress. The inhibition of H₂O₂-induced IL-8 secretion from Caco-2 cells was observed by pretreatment with Cys, Val, Ile, Leu, Trp, His, Lys, and Ala. Cys enhanced glutathione (GSH) biosynthesis enzyme activity and increased cellular GSH levels. Branched-chain amino acids such as Val, Ile, and Leu elevated activities of GSH S-transferase (GST) and catalase. Trp, His, and Lys caused increases in GST activity. Ala enhanced GSH reductase activity. These data suggest that specific amino acids exert protective effects against tissue oxidative stress in intestinal epithelial cells.

Peptides derived from seaweed pipefish Syngnathus schlegeli SPP-1(QLGNLGV) and SPP-2 (SVMPVVA) were assessed for their ability to prevent H₂O₂ induced oxidative stress in human dermal fibroblasts (HDFs). Both peptides showed a significant hydroxyl radical scavenging activity when tested by ESR technique. The peptides effectively suppressed H₂O₂ induced ROS production and DNA damage in HDF cells. Furthermore the two peptides increase the protein expression levels of intracellular antioxidant enzymes SOD1, GSH and catalase in H₂O₂ stressed HDF cells. At the cellular signaling level, SPPs block the NF-κB activation which may lead to the reduction of oxidative stress mediated damage of HDF cells. These findings indicate the potential antioxidant effects of SPPs as response to H₂O₂ stimulation. (Peptides Volume 32, Issue 4, April 2011, Pages 639-647).

In a study by Morifuji M, et al. (J Nutr Sci Vitaminol, 55, 81-86, 2009) to identify bioactive peptides in whey protein hydrolysates (WPH) that stimulated glucose uptake and glycogen synthesis rate in skeletal muscle, the study found that the BCAA-containing dipeptides, Ile-Val, Leu-Val, Val-Leu, Ile-Ile, Dipeptides and tripeptides, Ile-Leu, and Leu-Leu caused significant stimulation in glucose uptake rate in the L6 myotubes. Ile-Leu, the main component in WPH, also stimulated glucose uptake in isolated skeletal muscles. Stimulation of glucose uptake by Ile-Leu was completely inhibited by treatment with either LY294002, or GF109203X in both L6 cells and isolated muscles. Ile-Leu increased glycogen contents in isolated muscles. These results suggest that BCAA-containing bioactive dipeptides in WPH stimulate glucose uptake in skeletal muscles via the PI3-kinase and aPKC pathways, resulting in increased skeletal muscle glycogen contents.

A more recent study by Morato P. N. et al investigated which whey protein hydrolysate (WPH) component can modulate translocation of the glucose transporter GLUT-4 to the plasma membrane (PM) of animal skeletal muscle. The following WPH components were tested: Control, WPH, L-isoleucine, L-leucine, L-leucine plus L-isoleucine, L-isoleucyl-L-leucine dipeptide and L-leucyl-L-isoleucine dipeptide. Of the WPH components tested, the amino acid L-isoleucine and the peptide L-Leucyl-L-isoleucine showed greater efficiency in translocating GLUT-4 to the PM and of increasing glucose capture by skeletal muscle. (Food Chemistry 139 (2013) 853-859).

Recent evidence points to a strong relationship between increased mitochondrial biogenesis and increased survival in eukaryotes. BCAAs have been shown to extend life span in yeast. However, the role of these amino acids in mitochondrial biogenesis and longevity in mammals is unknown.

Here, we show that a BCAA-enriched mixture (BCAAem) increased the average life span of mice. BCAAem supplementation increased mitochondrial biogenesis and sirtuin 1 expression in primary cardiac and skeletal myocytes and in cardiac and skeletal muscle, but not in adipose tissue and liver of middle-aged mice, and this was accompanied by enhanced physical endurance. Moreover, the reactive oxygen species (ROS) defense system genes were upregulated, and ROS production was reduced by BCAAem supplementation. All of the BCAAem-mediated effects were strongly attenuated in endothelial nitric oxide synthase null mutant mice. These data reveal an important antiaging role of BCAAs mediated by mitochondrial biogenesis in mammals (Cell Metabolism, Volume 12, Issue 4, 362-372, 6 Oct. 2010).

A number of muscle-wasting and muscle-function impairment conditions have been identified, for which treatment with BCCAs has proved beneficial. For example, muscle fatigue and recovery has an impact on muscle damage and oxygen-carrying capacity. One beneficial effect of BCAA supplementation is a quicker recovery from the muscle fatigue that follows eccentric exercise training.

BCCAs can be oxidized in skeletal muscle. This phenomenon is attributed to activation of the branched-chain α-keto acid dehydrogenase (BCKDH) complex. This enzyme complex is regulated by a phosphorylation-dephosphorylation cycle. Administration of ligands for peroxisome proliferator-activated receptor-α (PPAR-α) causes activation of the hepatic BCKDH complex in association with a decreased kinase activity. Long-chain fatty acids are ligands for PPAR-α, and the fatty acid oxidation is promoted by several physiological conditions including exercise. This implies that fatty acids may be one of the regulators of BCAA catabolism and that BCAA requirement is increased by exercise. It follows that BCAA supplementation before and after exercise has beneficial effects for decreasing exercise-induced damage and promoting muscle-protein synthesis.

Food deprivation can cause a decrease in muscle protein synthesis in skeletal muscle. This blunting of protein synthesis is partially regulated by reduced signaling through mTORC1; furthermore food deprivation slows mTORC1 signaling activity, which negatively impacts both the initiation and elongation phases of mRNA translation, thereby resulting in reduced protein synthsis. Morato P. N, et al. have shown that supplementation with L-leucyl-isoleucine causes activation of Akt (Morato P. N et al Food Chemistry 139 (2013) 853-859). Activation of Akt causes activation of mTORC1. Activation of mTORC1 is known to activate at least nine other targets involved in muscle protein sysnthesis (Ruvinsky, et al. Trends Biochem Sci 2006, 31:342-48).

During oxidative stress, a spectrum of cellular derangements developed, including membrane phospholipid peroxidation, thiol oxidation, consumption of the major chain-breaking membrane antiperoxidant (alpha-tocopherol), and ATP loss. Branched-chain amino acids such as Val, Ile and Leu elevate activities of GSH-S-transferase (GST) and catalase suggesting that specific amino acids exert protective effects against tissue oxidative stress.

The wasting conditions and impairments discussed above are just a few of the disorders and limitations for which branched-chain amino acids supplementation has proved beneficial. BCAA supplementation has also been suggested to treat diabetes (Layman and Walker, J., Nutr. January 2006 vol. 136 no. 1 319S-323S); hepatic encephalopathy (Holecek, Nutrition, Volume 26, Issue 5, May 2010, Pages 482-490); serum cholesterol and triglycerides (Vianna, et al., Nutrition, Volume 28, Issue 2, February 2012, Pages 182-189); severe traumatic brain injury (Aquilani, et al., Archives of Physical medicine and Rehabilitation, Volume 86, Issue 9, September 2005, Pages 1729-1735); etc.

Pept-1 or SLC15A1 a solute carrier for oligopeptides, is localized to the brush border membrane of the intestinal epithelium and mediates the uptake of di- and tripeptides from the lumen into the enterocytes. An article by Adibi SA on the oligopeptide transporter Pept-1 indicates that the characterization of its function by in vivo and in vitro studies has shown that it transports dipeptides and tripeptides, but not free amino acids or peptides with more than three amino acid residues (Gastroenterology. 1997 July; 113(1):332-40.). In addition, an article in Digestion 60:332-337 (1999) that discusses “Effect of Chain Length on Absorption of Biologically Active Peptides from the Gastrointestinal Tract” concludes that: The potency of the enterally administered peptides decreases as the chain length increases.

Accordingly, there is a need in the art of orally-administrable nutritional products to provide functional dipeptides, tripeptides, and their derivatives that can prevent, treat, and/or ameliorate the conditions described above. Other needs that the present disclosure should also satisfy are that the orally-administrable nutritional products can serve as a viable alternative to performance-enhancing drugs (PED) to athletes.

### BRIEF SUMMARY OF THE INVENTION

Disclosed is an orally-administrable nutritional product including a dipeptide and/or a tripeptide that comprises a branched chain amino acid (BCAA) and/or its esters or ester hydrochlorides. In specific embodiments, the nutritional product can comprise a dipeptide or a tripeptide selected from L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine, or esters and or ester hydrochlorides thereof.

In one embodiment, a method for eliciting a beneficial effect in mammals includes ingesting or otherwise administering dipetides and/or tripeptides such as L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and/or L-Isoleucyl-L-Leucyl-L-leucine and their derivatives.

In another embodiment, methods of ameliorating muscle wasting diseases such as muscular dystrophy, cachecia, cancer, HIV, immunodeficiency syndrome; reversal of age; accelerated recovery from injury include administration of nutritional supplements and pharmaceutical dosage forms may be, for example, tablet or capsule dosage forms, aqueous and emulsion injectable formulations, and gel systems.

In another embodiment, methods of supportive care to enhance quality of life; and other related frailty syndrome conditions (sarcopenia, muscle weakness) include administration of nutritional supplements and pharmaceutical dosage forms may be, for example, tablet or capsule dosage forms, aqueous and emulsion injectable formulations, and gel systems.

In another embodiment, methods of decreasing whole-body protein degradation in Duchenne muscular dystrophy (DMD) include administration of nutritional supplements and pharmaceutical dosage forms may be, for example, tablet or capsule dosage forms, aqueous and emulsion injectable formulations, and gel systems.

In another embodiment, methods of preventing the development of colonic premalignancies in obesity-related colon cancer associated with hyperlipidemia and hyperinsulinemia include administration of nutritional supplements and pharmaceutical dosage forms may be, for example, tablet or capsule dosage forms, aqueous and emulsion injectable formulations, and gel systems.

In another embodiment, methods of preventing the development of colonic premalignancies in obesity-related colon cancer associated with hyperlipidemia and hyperinsulinemia include administration of nutritional supplements and pharmaceutical dosage forms may be, for example, tablet or capsule dosage forms, aqueous and emulsion injectable formulations, and gel systems.

In another embodiment, methods of enhancing cognitive recovery in cases of dementia syndrome (Alzheimer's disease, vascular dementia, frontotemporal dementia, semantic dementia and dementia with Lewy bodies such as in Parkinson's disease) include administration of nutritional supplements and pharmaceutical dosage forms may be, for example, tablet or capsule dosage forms, aqueous and emulsion injectable formulations, and gel systems.

In another embodiment, methods of increase of muscle mass by activation of protein kinase Akt, phosphorylated on the serine 473 residue. This protein, upon phosphorylation by insulin signalisation Akt activation causes mTOR activation which regulates muscle growth/hypertrophy (Bodine S et al. Nat Cell Biol, 3:1014-19; improves physical resistance in mammals subjected to physical exhaustion, reduces in muscle injury during competition, contributes to protection against stress, increases in muscle fatty acids for use as energy source during exercise, increases capacity to recover the glycogen levels in liver and skeletal muscle after exercise, stimulates of the expression of SIRT genes thus improving insulin sensitivity; induces the expression of the Angiotensin Converting Enzyme (ACE) genes thus increases blood pressure by causing blood vessels to constrict triggering higher maximum oxygen uptake (VO2max), associated with endurance in long distance sports events; induces the expression of DAF-16 genes, a group of genes that drive the biological processes involved in ageing, immunity and responses to physical or environmental stresses. The method includes administration of nutritional supplements and pharmaceutical dosage forms may be, for example, tablet or capsule dosage forms, aqueous and emulsion injectable formulations, and gel systems.

In another embodiment, a synergistic combination of dipeptides and/or tripeptides and/or their derivatives with anti-aging compounds that activate SIRT genes, ACE genes, and/or transcription factors such as DAF 16 may be administered in the form of a tablet or capsule, aqueous and emulsion injectable formulations, and gel systems.

In another embodiment, the invention contemplates the use of the ingredients listed below, although other ingredients that induce the expression of the aforementioned genes also are contemplated. These compounds include, for example, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

In another embodiment, a composition comprises an orally-administrable nutritional product comprising a dipeptide, and a tripeptide including a branched chain amino acid (BCAA).

In another embodiment, a composition comprises an orally-administrable nutritional product comprising derivatives of these dipeptides and tripeptides such as the esters and esters hydrochlorides.

In another embodiment, dipeptides and tripeptides that can be used in the composition include L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine and their derivatives Ethyl esters and Ethyl Ester Hydrochlorides.

In another embodiment, a composition comprises orally-administrable synergistic combinations of the dipeptides and or tripeptides and their derivatives when other anti-aging compounds that activate SIRT genes, ACE genes and/or transcription factors such as DAF 16 are present. These synergistic ingredients that induce the expression of the aforementioned genes also become part of this disclosure. These ingredients include, for example, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Disclosed is an orally-administrable nutritional product comprising a dipeptide and/or a tripeptide and optionally including a branched chain amino acid (BCAA) and its esters and ester hydrochlorides. The nutritional product may comprise a dipeptide or a tripeptide selected from at least L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine or its derivative esters and or esters hydrochlorides.

Dipetides and tripeptides, including L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine and derivatives thereof, may elicit a beneficial effect when ingested by mammals or administered otherwise.

Also disclosed are nutritional supplements and pharmaceutical dosage forms, in tablet or capsule dosage forms, aqueous and emulsion injectable formulations, gel systems that can be used for: ameliorating muscle wasting diseases such as muscular dystrophy, cachecia, cancer, HIV, immunodeficiency syndrome; reversal of age; accelerated recovery from injury; supportive care to enhance quality of life and other related frailty syndrome conditions (sarcopenia, muscle weakness); decreasing whole-body protein degradation in Duchenne muscular dystrophy (DMD); preventing the development of colonic premalignancies in obesity-related colon cancer associated with hyperlipidemia and hyperinsulinemia; enhancing cognitive recovery in cases of dementia syndrome (Alzheimer's disease, vascular dementia, frontotemporal dementia, semantic dementia, and dementia with Lewy bodies such as in Parkinson's disease); increasing of muscle mass by activation of protein kinase Akt, phosphorylated on the serine 473 residue. This protein being phosphorylated by insulin signalisation Akt activation causes mTOR activation which regulates muscle growth/hypertrophy (Bodine S et al. Nat Cell Biol, 3:1014-19; improved physical resistance in mammals subjected to physical exhaustion, reduced muscle injury during competition, contributed to protection against stress, increased muscle fatty acids for use as energy source during exercise, increased capacity to recover the glycogen levels in liver and skeletal muscle after exercise, stimulation of the expression of SIRT genes thus improving insulin sensitivity; induced the expression of the Angiotensin Converting Enzyme (ACE) genes thus increasing blood pressure by causing blood vessels to constrict triggering higher maximum oxygen uptake (VO2max), is associated with endurance in long distance sports events; induced the expression of DAF-16 genes, which are a group of genes that drive the biological processes involved in aging, immunity and responses to physical or environmental stresses.

Additionally, synergistic combinations of the peptides L-leucine and L-isoleucine (dipeptides and tripeptides) and derivatives when used with other anti-aging compounds may activate SIRT genes, ACE genes and/or transcription factors such as DAF 16. The disclosure includes the ingredients listed below, although other ingredients that induce the expression of the aforementioned genes also become part of this disclosure. These compounds are: ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

Descriptions of the Orally-Administrable Nutritional Products

An oral liquid composition (Ready-to-Drink) comprising dipeptides and/or tripeptides and/or derivatives, said composition with a pH of about 3.5 to 6.5 being substantially stable at room temperature for normal warehouse storage conditions. The composition comprises a suitable aqueous solvent or vehicle, a non-aqueous vehicle, a preservative, a physical stabilizing ingredient, one or more surfactants, and one or more buffer salts that can render the composition pH stable.

The composition may also contain creatine, creatine derivatives and/or creatylated peptides as described in U.S. Pat. No. 8,445,466, which is incorporated herein in its entirety by reference, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein. The composition may also contain nucleosides, oligonucleotides, monophosphates, diphosphates, triphosphates and cyclic derivatives of these nucleosides (ATP, UTP, etc.), and amino acids, vitamins and vitamin-like isoprenoids, peptides and one or more additional components selected from the group consisting of: lipids, short, medium and long chain triglycerides, starches, carbohydrates, polyols, minerals, electrolytes, amino trace elements, colorings, flavors, artificial sweeteners, and anti-oxidants.

An oral liquid composition for buccal sublingual administration comprising dipeptides and/or tripeptides and/or derivatives, said composition with a pH of about 3.5 to 6.5 being substantially stable at room temperature for normal warehouse storage conditions. The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein. The composition comprises a suitable aqueous solvent or vehicle, a non-aqueous vehicle, a preservative, a physical stabilizing ingredient, one or more surfactants, and one or more buffer salts that can render the composition pH stable.

The composition may also contain nucleosides, oligonucleotides, the monophosphates, diphosphates, triphosphates and cyclic derivatives of these nucleosides (ATP, UTP, etc.), and amino acids, vitamins and vitamin-like isoprenoids, peptides and one or more additional components selected from the group consisting of: lipids, short, medium and long chain triglycerides, starches, carbohydrates, polyols, minerals, electrolytes, amino trace elements, colorings, flavors, artificial sweeteners, and anti-oxidants.

An oral solid composition in the form of a capsule (e.g. LiCap®) with a liquid composition as fill material containing dipeptides and/or tripeptides and/or their derivatives, said liquid fill material being substantially stable at room temperature for normal warehouse storage conditions. The composition further comprises a suitable lipophilic solvent or vehicle, a hydrophilic non-aqueous vehicle, a preservative, a physical stabilizing ingredient, one or more surfactants, and one or more buffer salts that can render the composition pH stable.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

The composition may also contain nucleosides, oligonucleotides, the monophosphates, diphosphates, triphosphates and cyclic derivatives of these nucleosides (ATP, UTP, etc.), and amino acids, vitamins and vitamin-like isoprenoids, peptides and one or more additional components selected from the group consisting of: lipids, medium and short chain triglycerides, starches, polyols, carbohydrates, minerals, electrolytes, amino trace elements, colorings, and anti-oxidants.

An oral liquid composition containing from 1 gram to 100 grams of protein and from 1 gram to 100 grams of carbohydrates per serving comprising dipeptides and/or tripeptides and/or their derivatives, said liquid being substantially stable at room temperature for normal warehouse storage conditions.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

The composition comprises an acid stable protein isolate, or a combination or blend of protein isolates, concentrates and hydrolyzates and caseins in micellar forms, a suitable aqueous solvent or vehicle, a non-aqueous vehicle, a preservative, a physical stabilizing ingredient, one or more surfactants, and one or more buffer salts that can render the composition pH stable.

The composition may also contain nucleosides, oligonucleotides, the monophosphates, diphosphates, triphosphates and cyclic derivatives of these nucleosides (ATP, UTP, etc.), and amino acids, vitamins and vitamin-like isoprenoids, peptides and one or more additional components selected from the group consisting of: lipids, short, medium and long chain triglycerides, starches, carbohydrates, polyols, minerals, electrolytes, amino trace elements, colorings, flavors, artificial sweeteners, and anti-oxidants.

An aqueous injectable composition for human administration said composition being isotonic and sterile in nature comprising dipeptides and/or tripeptides and/or their derivatives, said injectable preparation with a pH of about 3.5, being substantially stable at room temperature for normal warehouse storage conditions.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, and the lactotripeptides Val-Pro-Pro and Ile-Pro-Pro produced by the probiotic Lactobacillus helveticus or derived from casein.

The composition further comprises a suitable aqueous solvent, a preservative, a physical stabilizing ingredient and one or more buffer salts that can render the composition pH stable.

The composition may also contain nucleosides, oligonucleotides, the monophosphates, diphosphates, triphosphates and cyclic derivatives of these nucleosides (ATP, UTP, etc.), and amino acids, peptides, proteins and carbohydrates.

An emulsion injectable composition for human administration said composition being isotonic and sterile in nature may comprise dipeptides and/or tripeptides and/or its derivatives, said injectable preparation with a pH of about 3.5, being substantially stable at room temperature for normal warehouse storage conditions.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

The composition may further comprise a suitable aqueous solvent, pharmaceutically acceptable oils (sesame, olive, castor, peanut, cotton seed, short, medium and long chain triglycerides, etc.), a natural emulsifier such as lecithin or any other synthetic emulsifier, be it of the polysorbate or ethoxylated glyceride type, a preservative, a physical stabilizing ingredient and one or more buffer salts that can render the composition pH stable.

The composition may also contain nucleosides, oligonucleotides, the monophosphates, diphosphates, triphosphates and cyclic derivatives of these nucleosides (ATP, UTP, etc.), and amino acids, peptides, proteins and carbohydrates.

A gel topical composition for skin application in humans and animals said composition being clear or slightly opaque and having a gel consistency so that it can be spread on a skin surface, may comprise dipeptides and/or tripeptides and/or derivatives, said gel with a pH of about 3.5 to 6.5 being substantially stable at room temperature for normal warehouse storage conditions.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, the lactotripeptides Val-Pro-Pro and Ile-Pro-Pro produced by the probiotic Lactobacillus helveticus or derived from casein.

The composition may further comprise a suitable aqueous solvent, a preservative, a polymer for imparting consistency, a physical stabilizing ingredient and one or more buffer salts that can render the composition pH stable.

The composition may also contain nucleosides, oligonucleotides, the monophosphates, diphosphates, triphosphates and cyclic derivatives of these nucleosides (ATP, UTP, etc.), and amino acids, vitamins and vitamin-like isoprenoids, peptides, proteins and carbohydrates.

A cream topical composition for skin application in humans and animals said composition being an emulsion system or an opacified gel system and having a creamy consistency so that it can be spread on a skin surface and comprising dipeptides and/or tripeptides and/or derivatives, said cream with a pH of about 3.5 to 6.5 being substantially stable at room temperature for normal warehouse storage conditions.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, and the lactotripeptides Val-Pro-Pro and Ile-Pro-Pro produced by the probiotic Lactobacillus helveticus or derived from casein.

The composition may further comprise a suitable aqueous solvent, a preservative, a physical stabilizing ingredient, a surfactant, moisturizers, and one or more buffer salts that can render the composition pH stable.

The composition may also contain nucleosides, oligonucleotides, the monophosphates, diphosphates, triphosphates and cyclic derivatives of these nucleosides (ATP, UTP, etc.), and amino acids, vitamins and vitamin-like isoprenoids, peptides and one or more additional components selected from the group consisting of: lipids, short, medium and long chain triglycerides, starches, carbohydrates, polyols, minerals, electrolytes, amino trace elements, colorings, flavors, artificial sweeteners, and anti-oxidants.

A deep-penetrating transdermal composition for application in humans and animals said composition being a solution, a gel-like or an emulsion-like system or an opacified gel-like system and having a consistency so that it can be spread on a skin surface and comprising dipeptides and/or tripeptides and/or derivatives, said transdermal composition being substantially stable at room temperature for normal warehouse storage conditions.

The composition may also contain creatine, creatine derivatives and/or creatylated peptides as described in U.S. Pat. No. 8,445,466, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, and the lactotripeptides Val-Pro-Pro and Ile-Pro-Pro produced by the probiotic Lactobacillus helveticus or derived from casein.

The composition comprises a suitable aqueous solvent, a non-aqueous solvent, one or more penetrating enhancers, a preservative, a physical stabilizing ingredient, one or more surfactants, moisturizers, and one or more buffer salts that can render the composition pH stable. The composition may also contain nucleosides, oligonucleotides, the monophosphates, diphosphates, triphosphates and cyclic derivatives of these nucleosides (ATP, UTP, etc.), and amino acids, vitamins and vitamin-like isoprenoids, peptides, proteins, lipids, short, medium and long chain triglycerides and carbohydrates.

A transdermal patch delivery system comprising a liner, an adhesive, a backing and an aqueous liquid reservoir composition. The aqueous liquid reservoir composition being a solution or a suspension comprising dipeptides and/or tripeptides and/or derivatives, said transdermal patch being substantially stable at room temperature for normal warehouse storage conditions.

The composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins and lactokinins, breakdown products of casein and whey, and the lactotripeptides Val-Pro-Pro and Ile-Pro-Pro produced by the probiotic Lactobacillus helveticus or derived from casein.

The composition comprises a suitable aqueous solvent, a non-aqueous solvent, one or more penetrating enhancers, a preservative, a physical stabilizing ingredient, one or more surfactants, and one or more buffer salts that can render the composition pH stable.

The composition may also contain nucleotides, oligonucleotides, the monophosphates, diphosphates, triphosphates and cyclic derivatives of these nucleotides, and amino acids, vitamins and vitamin-like isoprenoids, peptides, proteins and carbohydrates.

A composition as provided herein may be administered chronically. As used herein, “chronically” has its normal meaning, which generally means repeated ingestion or application over a period of several days, several weeks or even several months. “Chronic” is generally not acute.

Consideration must be given to the fact that although this disclosure has been described and disclosed in relation to certain preferred embodiments, obvious equivalent modifications, and alterations thereof will become apparent to one of ordinary skill in this art upon reading and understanding this specification and the claims appended hereto. This includes subject matter defined by any combination of any one of the various claims appended hereto with any one or more of the remaining claims, including the incorporation of the features and/or limitations of any dependent claim, singly or in combination with features and/or limitations of any one or more of the other dependent claims, with features and/or limitations of any one or more of the independent claims, with the remaining dependent claims in their original text being read and applied to any independent claims so modified. This also includes combination of the features and/or limitations of one or more of the independent claims with features and/or limitations of another independent claim to arrive at a modified independent claim, with the remaining dependent claims in their original text being read and applied to any independent claim so modified. Accordingly, the present disclosure is intended to cover all such modifications and alterations and is not intended to be necessarily limited by any one or more particular strict interpretations of the claims which now follow.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### EXAMPLES

Following are examples which illustrate embodiments and procedures for practicing the invention. These examples should not be construed as limiting.

Ready to Drink Formulation

### Example 1

| INGREDIENTS (Ready-to-Drink Formulation) | % w/w |
|---|---|
| Purified water | 97.1 |
| Dipeptides and/or tripeptides and/or derivatives | 1.00 |
| Gamma Butyrobetaine | 0.0156 |
| Glycerin | 1.067 |
| Anserine | 0.052 |
| Caffeine | 0.06 |
| Magnesium Tanshinoate | 0.0000009 |
| L-Leucine | 0.104 |
| L-Isoleucine | 0.052 |
| L-Valine | 0.0208 |
| 1,3-di-n-propyl-7-propargylxanthine | 1E-10 |
| Citric acid to pH 3.5 | 0.179 |
| Sodium benzoate | 0.052 |
| Potassium sorbate | 0.01 |
| Bis picolinate vanadium | 0.0000002 |
| Salt | 0.005 |
| Potassium phosphate dibasic | 0.0206 |
| Sodium Erythorbate | 0.000001 |
| Nisaplin | 0.000001 |
| Sucralose | 0.073 |
| Malic acid | 0.083 |
| Flavor Melon | 0.105 |

### Liquid Buccal Sublingual Solution

### Example 2

| INGREDIENTS | % w/w |
|---|---|
| Dipeptides and/or tripeptides and/or derivatives | 2.10 |
| Peptides | 3.00 |
| AMP | 12.50 |
| UTP | 0.10 |
| Alcohol USP | 45.0 |
| Buffer Salt(s) | QS to adjust pH |
| Purified Water | QS to 100 |

### Example 3

| INGREDIENTS | % w/w |
|---|---|
| Dipeptides and/or tripeptides and/or derivatives | 2.10 |
| Peptides | 3.00 |
| AMP | 12.50 |
| UTP | 0.10 |
| Alcohol USP | 50.0 |
| Benzyl alcohol | 1.00 |
| Buffer Salt(s) | QS to adjust pH |
| Purified Water | QS to 100 |

### Example 4

| INGREDIENTS | % w/w |
|---|---|
| Dipeptides and/or tripeptides and/or derivatives | 2.10 |
| Peptides | 3.00 |
| AMP | 12.50 |
| UTP | 0.10 |
| Propylene Glycol | 20.0 |
| Alcohol USP | 40.0 |
| Polysorbate 80 | 5.0 |
| Benzyl alcohol | 1.00 |
| Buffer Salt(s) | QS to adjust pH |
| Purified Water | QS to 100 |

### Fill Material Composition for Capsule

### Example 5

| INGREDIENTS | % w/w |
|---|---|
| Dipeptides and/or tri- | 2.10 |
| peptides and/or | |
| derivatives | |
| Medium chain | 15.0 |
| triglyceride | |
| Peptides | 3.00 |
| AMP | 12.50 |
| UTP | 0.10 |
| Oleic Acid | 52.0 |

### Example 6

| INGREDIENTS | % w/w |
|---|---|
| Dipeptides and/or tri- | 2.10 |
| peptides and/or | |
| derivatives | |
| Peptides | 3.00 |
| AMP | 12.50 |
| UTP | 0.10 |
| Polysorbate 80 | 25.0 |
| PEG-40 Hydrogenated | 38.00 |
| Castor Oil | |
| PEG esters and mono- | 15.0 |
| glycerides | |

### Example 7

| INGREDIENTS | % w/w |
|---|---|
| Dipeptides and/or tri- | 2.10 |
| peptides and/or | |
| derivatives | |
| Peptides | 3.00 |
| AMP | 12.50 |
| UTP | 0.10 |
| PEG-400 | 45.0 |
| PEG esters and mono- | 9.00 |
| glycerides | |
| Polysorbate 80 | 20.0 |

### Medium Range pH RTD Protein Blend Formulations

### Example 8

| | % | Per 16 oz |
|---|---|---|
| INGREDIENTS | w/w | Serving |
| Dipeptides and/or tri- | 1.00 | 2.10 |
| peptides and/or | | |
| derivatives | | |
| Whey Protein Isolate | 6.000 | 30.00 |
| Whey Protein Concentrate | 0.640 | 3.20 |
| Whey Hydrolysate | 0.320 | 1.60 |
| Micellar casein | 0.320 | 1.60 |
| Casein Protein Hydrolysate | 0.000 | 0.00 |
| Potassium Chloride | 0.076 | 0.38 |
| Ascorbic Acid | 0.012 | 0.06 |
| Vitamin E TPGS | 0.052 | 0.26 |
| Riboflavin 100 | 0.000 | 0.00000010 |
| Niacin | 0.000 | 0.0020 |
| Pyrodoxine HC1 | 0.000 | 0.000007 |
| Calcium Panthothenate | 0.000 | 0.0011 |
| Magnesium Maleate | 0.020 | 0.1000 |
| d-ribose | 0.040 | 0.2000 |
| Lecithin | 0.600 | 3.00 |
| Saflower Oil | 1.200 | 6.00 |
| Sunflower Oil | 1.200 | 6.00 |
| Medium Chain Triglycerides | 0.800 | 4.00 |
| L-Glutamine | 0.025 | 0.13 |
| Glucose Polymers (Rice trin) | 0.800 | 4.00 |
| Waxy Maize Starch | 1.000 | 5.00 |
| High Amylose Starch | 0.100 | 0.50 |
| (Amylose ADP11P) | | |
| Magnesium Citrate | 0.124 | 0.62 |
| Microcrystalline Cellulose | 0.100 | 0.50 |
| Malic Acid | 0.140 | 0.70 |
| Citric acid to pH 6.5 | 0.566 | 2.83 |
| Sodium Citrate to pH 6.5 | 0.140 | 0.70 |
| Sucralose | 0.011 | 0.06 |
| Glycerin | 3.000 | 15.00 |
| Sodium Benzoate | 0.090 | 0.45 |
| Potassium Sorbate | 0.190 | 0.95 |
| Water | QS | QS |

### Example 9

| | mg/ |
|---|---|
| Ingredient | 16 oz |
| Dipeptide or tripeptide | 1000 |
| L-Leucine | 4000 |
| L-Isoleucine | 4000 |
| L-Valine | 2000 |
| Purified Water | 464561 |
| Citric Acid Anhydrous | 1000 |
| Sodium Benzoate | 250 |
| Potassium Sorbate FCC | 350 |
| EDTA-Calcium Di- | 225 |
| sodium Complex | |
| Malic Acid | 4 |

### Low pH RTD Protein Formulations

### Example 10

| | Per 16 oz | % |
|---|---|---|
| INGREDIENTS | serving | w/w |
| Dipeptides and/or tri- | 2.10 | 0.25 |
| peptides and/or | | |
| derivatives | | |
| Whey Protein Isolate | 44.44 | 9.26 |
| Acid Stable | | |
| Sucralose | 0.12 | 0.025 |
| Sodium EDTA | 0.24 | 0.050 |
| Potassium Sorbate | 0.96 | 0.200 |
| Sodium Benzoate | 0.48 | 0.100 |
| Citric Acid to pH 3.5 | QS | QS |
| Malic Acid to pH 3.5 | QS | QS |
| Water | 433.8 | 90.37 |
| | 480 | 100 |

### Low pH RTD Protein Formulations

### Example 11

| | Per 16 oz | % |
|---|---|---|
| INGREDIENTS | serving | w/w |
| Dipeptides and/or tri- | 2.10 | 0.25 |
| peptides and/or | | |
| derivatives | | |
| Whey Protein Isolate | 44.44 | 9.26 |
| Acid Stable | | |
| Sucralose | 0.12 | 0.025 |
| Waxy Maize Starch | 4.80 | 1.00 |
| Glucose Polymers | 0.96 | 0.20 |
| (Rice trin) | | |
| Na EDTA | 0.24 | 0.050 |
| Potassium Sorbate | 0.96 | 0.200 |
| Sodium Benzoate | 0.48 | 0.100 |
| Citric Acid to pH 3.5 | QS | QS |
| Malic Acid to pH 3.5 | QS | QS |
| Water | QS | QS |
| TOTAL | 480 | 100 |

### Aqueous Injectable Formulations

### Example 12

| | % |
|---|---|
| INGREDIENTS | w/v |
| Dipeptides and/or tri- | 1.00 |
| peptides and/or | |
| derivatives | |
| AMP | 12.5 |
| UTP | 0.10 |
| Amino Acids | 3.0-7.0 |
| Polysorbate 80 | 0.40 |

| | |
|---|---|
| Sodium CMC | 0.50 |
| Sodium Chloride | 0.90 |
| Benzyl alcohol | 0.90 |
| Buffer Salt(s) | QS to adjust to |
| | pH 3.5--6.5 |
| Sodium Hydroxide | QS to adjust to |
| | pH 3.5-6.5 |
| Water for Injection | QS to 100 |

### Example 13

| INGREDIENTS | % w/v |
|---|---|
| Dipeptides and/or tri- | 1.00 |
| peptides and/or | |
| derivatives | |
| AMP | 12.50 |
| UTP | 0.10 |
| Amino Acids | 3.0-7.0 |
| Polysorbate 80 | 0.40 |
| Sorbitol | 40.00 |
| Sodium Chloride | 0.90 |
| Benzyl alcohol | 0.90 |
| Buffer Sat(s) | QS to adjust to pH 3.5--6.5 |
| Sodium Hydroxide | QS to adjust to pH 3.5-6.5 |
| Water for Injection | QS to 100 |

### Example 14

| INGREDIENTS | % w/v |
|---|---|
| Dipeptides and/or tri- | 2.10 |
| peptides and/or | |
| derivatives | |
| Polysorbate 80 | 0.40 |
| AMP | 12.50 |
| UTP | 0.10 |
| Amino Acids | 3.0-7.0 |
| Sodium Citrate | 0.50 |
| Sodium Chloride | 0.90 |
| Benzyl alcohol | 0.90 |
| Buffer Salt(s) | QS to adjust to pH 3.5--6.5 |
| Sodium Hydroxide | QS to adjust to pH 3.5-6.5 |
| Water for Injection | QS to 100 |

### Emulsion Injectable Formulations

### Example 14

| INGREDIENTS | % w/v |
|---|---|
| Dipeptides and/or tri- | 1.00 |
| peptides and/or | |
| derivatives | |
| AMP | 12.5 |
| UTP | 0.10 |
| Amino Acids | 3.0-7.0 |
| Sesame Oil or Glycerides | 2.0-12.0 |
| Polysorbate 80 | 0.40 |
| Sodium Chloride | 0.90 |
| Benzyl alcohol | 0.90 |
| Buffer Salt(s) | QS to adjust to pH 3.5--6.5 |
| Sodium Hydroxide | QS to adjust to pH 3.5-6.5 |
| Water for Injection | QS to 100 |

### Example 15

| INGREDIENTS | % w/v |
|---|---|
| Dipeptides and/or tri- | 1.00 |
| peptides and/or | |
| derivatives | |
| AMP | 12.50 |
| UTP | 0.10 |
| Amino Acids | 3.0-7.0 |
| Olive Oil or Glycerides | 1.0-15.0 |
| Lecithin | 0.50-5.0 |
| Sorbitol | 30.00 |
| Sodium Chloride | 0.90 |
| Benzyl alcohol | 0.90 |
| Buffer Sat(s) | QS to adjust to pH 3.5--6.5 |
| Sodium Hydroxide | QS to adjust to pH 3.5-6.5 |
| Water for Injection | QS to 100 |

### Example 16

| INGREDIENTS | % w/v |
|---|---|
| Dipeptides and/or tri- | 1.00 |
| peptides and/or | |
| derivatives | |
| Peanut Oil or Glycerides | 1.0-15.0 |
| Polysorbate 80 | 0.2-10.0 |
| AMP | 12.50 |
| UTP | 0.10 |
| Amino Acids | 3.0-7.0 |
| Sodium Citrate | 0.50 |
| Sodium Chloride | 0.90 |
| Benzyl alcohol | 0.90 |
| Buffer Salt(s) | QS to adjust to pH 3.5--6.5 |
| Sodium Hydroxide | QS to adjust to pH 3.5-6.5 |
| Water for Injection | QS to 100 |

### Gel Formulations

### Example 17

| INGREDIENTS | % w/w |
|---|---|
| Dipeptides and/or tri- | 1.00 |
| peptides and/or | |
| derivatives | |
| Peptides/Polypeptides | 3.00 |
| AMP | 12.50 |
| UTP | 0.10 |
| Propylene Glycol | 12.0 |
| Carbomer | 1.00 |
| Buffer Salt(s) | QS to pH 3.5-6.5 |
| Methylparaben | 0.20 |
| Propylparaben | 0.10 |
| Purified Water | QS to 100 |

### Example 18

| INGREDIENTS | % w/w |
|---|---|
| Dipeptides and/or tripeptides and/or derivatives | 1.00 |
| Peptides/Polypepetides | 3.00 |
| AMP | 12.50 |
| UTP | 0.10 |
| Glycerin | 5.00 |
| Hydroxyethylcellulose | 2.00 |
| Triethanolamine | QS to pH 3.5-6.5 |
| Methylparaben | 0.20 |
| Propylparaben | 0.10 |
| Purified Water | QS to 100 |

### Example 19

| INGREDIENTS | % w/w |
|---|---|
| Dipeptides and/or tripeptides and/or derivatives | 1.00 |
| Peptides/Polypepetides | 3.00 |
| AMP | 12.50 |
| UTP | 0.10 |
| Glycerin | 15.0 |
| Poloxamers 407/188 | 10.00 |
| Triethanolamine | QS to pH 3.5-6.5 |
| Methylparaben | 0.025 |
| Propylparaben | 0.015 |
| Purified Water | QS to 100 |

### Cream Formulations

### Example 20

| INGREDIENTS | % w/w |
|---|---|
| Dipeptides and/or tripeptides and/or derivatives | 1.00 |
| White Petrolatum | 20.0 |
| Stearyl Alcohol | 20.0 |
| Propylene Glycol | 12.0 |
| Peptides/Polypeptides | 3.00 |
| AMP | 12.50 |
| UTP | 0.10 |
| Sodium lauryl sulfate | 1.00 |
| Methylparaben | 0.20 |
| Propylparaben | 0.10 |
| Buffer Salt(s) | QS to pH 3.5-6.5 |
| Purified Water | QS to 100 |

### Example 21

| INGREDIENTS | % w/w |
|---|---|
| Dipeptides and/or tripeptides and/or derivatives | 1.00 |
| Peptides/Polypeptides | 3.00 |
| AMP | 12.50 |
| UTP | 0.10 |
| Mineral Oil | 15.0 |
| Lanolin Alcohol | 10.0 |
| Cetyl Alcohol | 0.20 |
| Beeswax | 4.00 |
| Sorbitan Monoleate | 5.00 |
| Glycerin | 5.00 |
| Borax | 0.30 |
| Triethanolamine | 0.70 |
| Methylparaben | 0.20 |
| Propylparaben | 0.10 |
| Buffer Salt(s) | QS to pH 3.5-6.5 |
| Purified Water | QS to 100 |

### Example 22

| INGREDIENTS | % w/w |
|---|---|
| Dipeptides and/or tripeptides and/or derivatives | 1.00 |
| Peptides/Polypeptides | 3.00 |
| AMP | 12.50 |
| UTP | 0.10 |
| Glyceryl Monostearate | 10.0 |
| Lanolin | 2.00 |
| Glycerin | 10.0 |
| Stearyl Pyridinium Chloride | 1.50 |
| Methylparaben | 0.025 |
| Propylparaben | 0.015 |
| Buffer Salt(s) | QS to pH 3.5-6.5 |
| Purified Water | QS to 100 |

### Transdermal Systems

### Example 23

| INGREDIENTS | % w/w |
|---|---|
| Dipeptides and/or tripeptides and/or derivatives | 5.00 |
| N-methylpyrrolidone | 15.0 |
| Peptides | 3.00 |
| AMP | 12.50 |
| UTP | 0.10 |
| Alcohol USP | 2.00 |
| Benzyl alcohol | 1.00 |
| Buffer Salt(s) | QS to pH 3.5-6.5 |
| Purified Water | QS to 100 |

### Example 24

| INGREDIENTS | % w/w |
|---|---|
| Dipeptides and/or tripeptides and/or derivatives | 5.00 |
| Peptides | 3.00 |
| AMP | 12.50 |
| UTP | 0.10 |
| Ethoxydiglycol | 25.0 |
| Alcohol USP | 2.00 |
| PEG esters and monoglycerides | 15.0 |
| Benzyl alcohol | 1.00 |
| Buffer Salt(s) | QS to pH 3.5-6.5 |
| Purified Water | QS to 100 |

### Example 25

| INGREDIENTS | % w/w |
|---|---|
| Dipeptides and/or tripeptides and/or derivatives | 5.00 |
| Peptides | 3.00 |
| AMP | 12.50 |
| UTP | 0.10 |
| Propylene Glycol | 25.0 |
| Alcohol USP | 4.00 |
| Polysorbate 80 | 10.0 |
| Benzyl alcohol | 1.00 |
| Buffer Salt(s) | QS to pH 3.5-6.5 |
| Purified Water | QS to 100 |

### Liquid Reservoir for Trandermal Patch

### Example 26

| INGREDIENTS | % w/w |
|---|---|
| Dipeptides and/or tripeptides and/or derivatives | 5.00 |
| N-methylpyrrolidone | 10.0 |
| Peptides | 3.00 |
| AMP | 12.50 |
| UTP | 0.10 |
| Alcohol USP | 45.0 |
| Benzyl alcohol | 1.00 |
| Buffer Salt(s) | QS to pH 3.5-6.5 |
| Purified Water | QS to 100 |

### Example 27

| INGREDIENTS | % w/w |
|---|---|
| Dipeptides and/or tripeptides and/or derivatives | 5.00 |
| Peptides | 3.00 |
| AMP | 12.50 |
| UTP | 0.10 |
| Ethoxydiglycol | 20.0 |
| Alcohol USP | 50.0 |
| Benzyl alcohol | 1.00 |
| Buffer Salt(s) | QS to pH 3.5-6.5 |
| Purified Water | QS to 100 |

### Example 28

| INGREDIENTS | % w/w |
|---|---|
| Dipeptides and/or tripeptides and/or derivatives | 5.00 |
| Peptides | 3.00 |
| AMP | 12.50 |
| UTP | 0.10 |
| Propylene Glycol | 20.0 |
| Alcohol USP | 40.0 |
| Polysorbate 80 | 5.0 |
| Benzyl alcohol | 1.00 |
| Buffer Salt(s) | QS to pH 3.5-6.5 |
| Purified Water | QS to 100 |

### Example 29

### Powder Blends

| Ingredients | mg/serving |
|---|---|
| Dipeptide or tripeptide | 500 |
| Creatyl-L-Leucine | 1000 |
| HICA (alpha hydroxy-isocaproic acid) | 1000 |
| L-Leucine | 1000 |
| Creatine Monohydrate | 2500 |
| L-Isoleucine | 1000 |
| Valine | 500 |
| Citric Acid Anhydrous | 800 |

### Example 30

### Powder Blends

| Ingredients | mg/serving |
|---|---|
| Dipeptide or tripeptide | 500 |
| L-Creatyl-L-Leucine | 1000 |
| Creatinol-0-Phosphate | 500 |
| L-Leucine | 1500 |
| HICA (alpha hydroxy-isocaproic acid) | 1000 |
| Creatine Monohydrate | 2500 |
| L-Isoleucine | 1500 |
| L-Valine | 750 |
| Caffeine Anhydrous | 250 |
| Theacrine | 50 |
| Trisodium Phosphate, Dodecahydrate | 250 |
| Pyridoxine HCl | 0.10 |
| MCT | 1000 |
| Citric Acid Anhydrous | 850 |

### Example 31

### Powder Blends

| Ingredients | Mg/serving |
|---|---|
| Dipeptide or tripeptide | 500 |
| L-Histidine | 5 |
| L-Isoleucine | 2500 |
| L-Leucine | 2500 |
| L-Lysine | 640 |
| L-Methionine | 90 |
| L-Phenylalanine | 900 |
| L-Threonine | 750 |
| L-Valine | 1250 |
| L-Citrulline Malate | 500 |
| Pyridoxine HCl | 5 |
| Citric Acid Anhydrous | 750 |

### Example 32

### Powder Blends

| INGREDIENTS | mg/servings |
|---|---|
| Dipeptide or tripeptide | 500 |
| Casein Protein Hydrolysate | 1500 |
| L-Leucine | 1200 |
| L-Isoleucine | 1200 |
| L-Valine | 600 |
| Vitamin D3 (Cholecalciferol) 1 Mio IU/G | 0.0005 |
| 1,25-dihydroxyvitamin D3 | 0.0125 |
| Potassium Citrate Monohydrate | 200 |
| Citric Acid Anhydrous | 400 |

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

What is claimed is:

1. An orally-administrable nutritional product comprising one or more dipeptides or tripeptides selected from the group consisting of L-Creatyl-L-Leucine, Creatyl-L-Leucine, L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine.

2. The orally-administrable nutritional product of claim 1 further comprising one or more anti-aging compounds selected from the group consisting of ephedrine, caffeine, omega-3 fatty acids (fish oil), butein, piceatannol, fisetin, quercetin, isoliquiritigenin, casokinins and lactokinins, casein, whey, lactotripeptides and creatine.

3. The orally-administrable nutritional product of claim 1 further comprising a nutritional adjuvant selected from the group consisting of flavoring agents, colorants, viscosity modifiers, preservatives, chelating agents, antioxidants, creatine and surface modifiers.

4. The orally-administrable nutritional product of claim 1 further comprising one or more ingredients selected from the group consisting of amino acids, vitamins, minerals, essential fatty acids, enzymes, mono-glycerides, di-glycerides, tri-glyceride ester oils, emulsifiers, hydrolyzed proteins, whey protein, creatine, stabilizers, flow modifiers, viscosity improvers, chelating agents, enzymes, nucleosides, oligonucleotides, monophosphates, diphosphates, triphosphates and surfactants.

5. The orally-administrable nutritional product of claim 1 wherein the product comprises a powder capable of being dissolved in an aqueous solution.

6. The orally-administrable nutritional product of claim 1 wherein the product comprises a liquid beverage suitable for consumption by humans.

7. The orally-administrable nutritional product of claim 5 further comprising: creatinol-0-phosphate;
one or more anti-aging compounds selected from the group consisting of ephedrine, caffeine, omega-3 fatty acids (fish oil), butein, piceatannol, fisetin, quercetin, isoliquiritigenin, casokinins and lactokinins, casein, whey, and actotripeptides;
one or more nutritional adjuvants selected from the group consisting of flavoring agents, colorants, viscosity modifiers, preservatives, chelating agents, antioxidants, creatine and surface modifiers;
one or more ingredients selected from the group consisting of amino acids, vitamins, minerals, essential fatty acids, enzymes, mono-glycerides, di-glycerides, tri-glyceride ester oils, emulsifiers, hydrolyzed proteins, whey protein, creatine, stabilizers, flow modifiers, viscosity improvers, chelating agents, enzymes, nucleosides, oligonucleotides, monophosphates, diphosphates, triphosphates and surfactants.

8. An orally-administrable nutritional product comprising one or more derivatives of one or more dipeptides or tripeptides selected from the group consisting of L-Creatyl-L-Leucine, Creatyl-L-Leucine, L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine.

9. The orally-administrable nutritional product of claim 8 wherein the derivatives of the one or more dipeptides or tripeptides are esters or ester hydrochlorides of the dipeptides or tripeptides.

10. The orally-administrable nutritional product of claim 8 wherein the derivatives of the one or more dipeptides or tripeptides are ethyl esters or ethyl ester hydrochlorides of the dipeptides or tripeptides.

11. The orally-administrable nutritional product of claim 8 further comprising one or more anti-aging compounds selected from the group consisting of ephedrine, caffeine, omega-3 fatty acids (fish oil), butein, piceatannol, fisetin, quercetin, isoliquiritigenin, casokinins and lactokinins, casein, whey, lactotripeptides and creatine.

12. The orally-administrable nutritional product of claim 8 further comprising a nutritional adjuvant selected from the group consisting of flavoring agents, colorants, viscosity modifiers, preservatives, chelating agents, antioxidants, creatine and surface modifiers.

13. The orally-administrable nutritional product of claim 8 further comprising amino acids, vitamins, minerals, essential fatty acids, enzymes, mono-glycerides, di-glycerides, tri-glyceride ester oils, emulsifiers, hydrolyzed proteins, whey protein, stabilizers, flow modifiers, viscosity improvers, chelating agents, enzymes, creatine, nucleosides, oligonucleotides, the monophosphates, diphosphates, triphosphates and surfactants.

14. A method for activating the expression of one or more of a SIRT gene, a ACE gene and a DAF 16 gene comprising the step of orally administering a composition comprising one or more dipeptides or tripeptides selected from the group consisting of L-Creatyl-L-Leucine, Creatyl-L-Leucine, L-leucyl-L-isoleucine, L-Leucyl-L-leucine, L-Isoleucyl-L-Leucine, L-Isoleucyl-L-isoleucine, L-Leucyl-L-isoleucyl-L-Leucine, L-leucyl-L-leucyl-L-isoleucine and L-Isoleucyl-L-Leucyl-L-leucine.

15. The method for activating the expression of one or more of a SIRT gene, a ACE gene and a DAF 16 gene of claim 14 wherein the orally-administrable nutritional product further comprises one or more anti-aging compounds selected from the group consisting of ephedrine, caffeine, omega-3 fatty acids (fish oil), butein, piceatannol, fisetin, quercetin, isoliquiritigenin, casokinins and lactokinins, casein, whey, lactotripeptides and creatine.

16. The method for activating the expression of one or more of a SIRT gene, a ACE gene and a DAF 16 gene of claim 14 wherein the orally-administrable nutritional product further comprises a nutritional adjuvant selected from the group consisting of flavoring agents, colorants, viscosity modifiers, preservatives, chelating agents, antioxidants, creatine and surface modifiers.

17. The method for activating the expression of one or more of a SIRT gene, a ACE gene and a DAF 16 gene of claim 14 wherein the orally-administrable nutritional product further comprises one or more ingredients selected from the group consisting of amino acids, vitamins, minerals, essential fatty acids, enzymes, mono-glycerides, di-glycerides, tri-glyceride ester oils, emulsifiers, hydrolyzed proteins, whey protein, creatine, stabilizers, flow modifiers, viscosity improvers, chelating agents, enzymes, nucleosides, oligonucleotides, monophosphates, diphosphates, triphosphates and surfactants.

18. The orally-administrable nutritional product of claim 14 wherein the product comprises a powder capable of being dissolved in an aqueous solution.

19. The orally-administrable nutritional product of claim 14 wherein the product comprises a liquid beverage suitable for consumption by humans.

20. The orally-administrable nutritional product of claim 18 further comprising creatinol-0-phosphate and:
one or more anti-aging compounds selected from the group consisting of ephedrine, caffeine, omega-3 fatty acids (fish oil), butein, piceatannol, fisetin, quercetin, isoliquiritigenin, casokinins and lactokinins, casein, whey, and lactotripeptides;
one or more nutritional adjuvants selected from the group consisting of flavoring agents, colorants, viscosity modifiers, preservatives, chelating agents, antioxidants, and surface modifiers; and,
one or more ingredients selected from the group consisting of amino acids, vitamins, minerals, essential fatty acids, enzymes, mono-glycerides, di-glycerides, tri-glyceride ester oils, emulsifiers, hydrolyzed proteins, whey protein, stabilizers, flow modifiers, viscosity improvers, chelating agents, enzymes, nucleosides, oligonucleotides, monophosphates, diphosphates, triphosphates and surfactants.
